(19) United States
(12) Patent Application Publication
(10) Pub. No.: US 2022/0362041 A1
(43) Pub. Date: Nov. 17, 2022

(54) FLOW DIVERTING DEVICE FOR DIALYSIS VASCULAR ACCESS AND METHODS FOR USE THEREWITH

(71) Applicant: David Namazy, San Diego, CA (US)

(72) Inventor: David Namazy, San Diego, CA (US)

(21) Appl. No.: 17/726,288

(22) Filed: Apr. 21, 2022

Related U.S. Application Data
(60) Provisional application No. 63/187,271, filed on May 11, 2021.

(51) Int. Cl. A61F 2/95; A61M 25/09; A61B 17/11; A61M 25/00

(52) U.S. Cl. CPC A61F 2/95; A61M 25/09; A61B 17/11; A61M 25/0068; A61M 2025/0681

(57) ABSTRACT

Embodiments of the disclosed technology provide flow diverting devices for dialysis vascular access, and methods for use therewith. According to some embodiments of the disclosed technologies, a medical device comprises: a tubular vascular stent; and a flow restrictor disposed within the tubular vascular stent. According to some embodiments of the disclosed technologies, a method comprises: providing a medical device, the medical device comprising: a tubular vascular stent, and a flow restrictor disposed within the tubular vascular stent; and deploying the medical device at a site within a vascular access of a patient. According to some embodiments of the disclosed technologies, a medical device comprises: a tubular vascular stent; one or more anchor struts formed on at least one end of the tubular stent; a flow restrictor disposed within the tubular vascular stent; and a tubular stent cover, wherein the vascular stent is disposed within the tubular stent cover.

### CROSS REFERENCE TO RELATED APPLICATIONS

The present application claims priority to U.S. Provisional Patent Application No. 63/187,271, filed May 11, 2021, entitled “FLOW DIVERTING DEVICE FOR DIALYSIS VASCULAR ACCESS,” the disclosure thereof incorporated by reference herein in its entirety.

### DESCRIPTION OF RELATED ART

The disclosed technology relates generally to medical devices, and more particularly some embodiments relate to vascular devices for use in dialysis.

### SUMMARY

In general, one aspect disclosed features a medical device comprising: a tubular vascular stent; and a flow restrictor disposed within the tubular vascular stent.

Embodiments of the vascular medical device may include one or more of the following features. Some embodiments comprise a tubular stent cover, wherein the vascular stent is disposed within the tubular stent cover. In some embodiments, the tubular stent comprises: one or more anchor struts formed on at least one end of the tubular stent. In some embodiments, the flow restrictor comprises: a longitudinal lumen, wherein a diameter of the lumen is uniform. In some embodiments, the flow restrictor comprises: a longitudinal lumen, wherein a diameter of the lumen is non-uniform. In some embodiments, the flow restrictor comprises: a longitudinal lumen, wherein a diameter of the lumen at an end of the lumen is greater than a diameter of the lumen between the ends of the lumen. In some embodiments, the flow restrictor comprises: a longitudinal lumen; and one or more projections extending into the lumen. Some embodiments comprise one or more radio-opaque markers.

In general, one aspect disclosed features a method comprising: providing a medical device, the medical device comprising: a tubular vascular stent, and a flow restrictor disposed within the tubular vascular stent; and deploying the medical device at a site within a vascular access of a patient.

Embodiments of the method may include one or more of the following features. In some embodiments, the vascular access is to a vein of the patient, and wherein disposing the medical device within a vascular access of a patient comprises: inserting a percutaneous needle in the retrograde direction into the vascular access; inserting a guidewire in the retrograde direction into the vascular access; inserting an angiographic sheath over the guidewire into the vascular access; inserting the medical device into the angiographic sheath over the guidewire into the vascular access; removing the sheath; and removing the guidewire. Some embodiments comprise performing a first arteriogram of the vascular access prior to deploying the medical device; selecting the site within the vascular access to deploy the medical device, an outer diameter of the tubular vascular stent, and a diameter of an inner lumen of the flow restrictor, based on the first arteriogram. Some embodiments comprise performing a second arteriogram subsequent to deploying the medical device; and evaluating blood flow through the vascular access, and blood flow through the artery distal to the anastomosis, based on the second arteriogram. In some embodiments, performing a first arteriogram comprises: inserting the guidewire in the retrograde direction into the vascular access and into an artery corresponding to the vein through an anastomosis between the artery and the vein such that a tip of the guidewire is located in the artery and proximal to the anastomosis; inserting a catheter along the guidewire such that a tip of the catheter is located in the artery and proximal to the anastomosis; and deploying arteriographic fluid in the artery and proximal to the anastomosis.

In general, one aspect disclosed features a medical device comprising: a tubular vascular stent; one or more anchor struts formed on at least one end of the tubular stent; a flow restrictor disposed within the tubular vascular stent; and a tubular stent cover, wherein the vascular stent is disposed within the tubular stent cover.

Embodiments of the medical device may include one or more of the following features. In some embodiments, the flow restrictor comprises: a longitudinal lumen, wherein a diameter of the lumen is uniform. In some embodiments, the flow restrictor comprises: a longitudinal lumen, wherein a diameter of the lumen is non-uniform. In some embodiments, the flow restrictor comprises: a longitudinal lumen, wherein a diameter of the lumen at an end of the lumen is greater than a diameter of the lumen between the ends of the lumen. In some embodiments, the flow restrictor comprises: a longitudinal lumen; and one or more projections extending into the lumen. Some embodiments comprise one or more radio-opaque markers.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 depicts vasculature of a patient's arm, including a portion of a vascular access, with the direction of blood flow indication by arrows.

FIG. 2 illustrates the vascular “steal phenomenon”.

FIG. 3 illustrates the formation of aneurysms due to venous hypertension.

FIG. 4 is a perspective view of a flow diverting device (FDD) according to some embodiments of the disclosed technologies.

FIG. 5A is a perspective view of a flow restrictor having an inner lumen of uniform diameter.

FIG. 5B is a cross-sectional view of the flow restrictor of FIG. 5A.

FIG. 6A is a perspective view of a flow restrictor having an inner lumen of non-uniform diameter.

FIG. 6B is a cross-sectional view of the flow restrictor of FIG. 6A.

FIG. 7A is a perspective view of a flow restrictor having a scalloped luminal structure.

FIG. 7B is a cross-sectional view of the flow restrictor of FIG. 7A.

FIG. 8 is a perspective view of a flow restrictor having a venous valve luminal structure.

FIG. 9 illustrates a portion of a deployment device according to some embodiments of the disclosed technologies.

FIG. 10 illustrates additional components of the deployment device of FIG. 9 according to some embodiments of the disclosed technologies.

FIG. 11 is a flowchart illustrating a process for deploying the FDD according to some embodiments of the disclosed technologies.

FIG. 12 illustrates a FDD deployed within a vascular access according to some embodiments of the disclosed technologies.

The figures are not exhaustive and do not limit the present disclosure to the precise form disclosed.

### DETAILED DESCRIPTION

Dialysis vascular access is a critical lifeline for patients on dialysis. Patients with kidney failure rely on dialysis to sustain life. Dialysis is the process of removal of toxins and excess sodium and water from the blood of a patient by a dialysis machine. In this process, blood flows from the patient's vascular access to the machine, and is cleaned by the machine, for example utilizing diffusion of solutes and water across a semipermeable membrane. The cleaned blood is then returned to the patient through the vascular access.

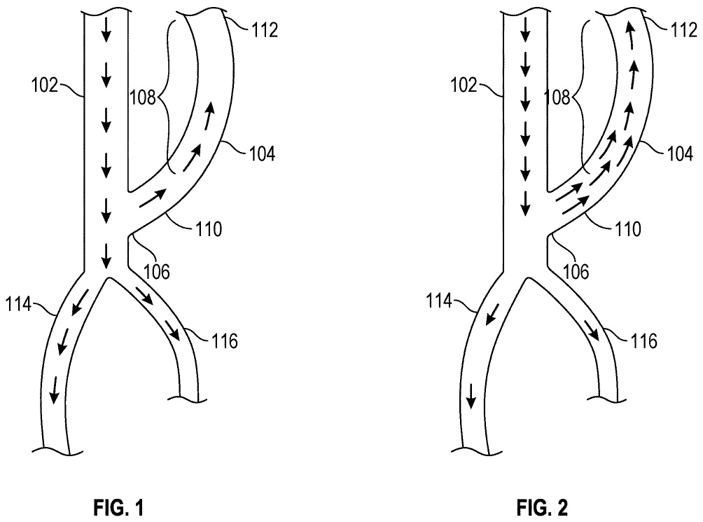

To facilitate dialysis, a vascular access is surgically created within the vasculature of the patient by connecting an artery with a vein. FIG. 1 depicts vasculature of a patient's arm with the direction of blood flow indication by arrows. In FIG. 1, the patient's brachial artery 102 and cephalic vein 104 have been connected surgically by a conduit referred to as an anastomosis 106. The most common vascular accesses currently in use are arteriovenous fistulas (AVF) and arteriovenous grafts (AVG). An AVF (fistula) is a connection between a native vein and artery. An AVG (graft) is an artificial tube connected to an artery on one end and a vein on the other. FIG. 1 depicts an AVF. However, it should be understood that the anastomoses described herein apply to AVFs, AVGs, and other types of anastomoses. Furthermore, while vascular access to the arm is described, the disclosed technology applies to other locations as well, including the thighs, the legs, and other locations.

After surgically creating the anastomosis 106, a vascular access is created for connection to the dialysis machine. The vascular access is generally created in the vein, and proximal to the anastomosis, so the cleaned blood returned to the patient flows toward the patient's heart for distribution throughout the patient's body. In FIG. 1, the vascular access 108 lies between the arterial end 110 of the vascular access 108 and the venous end 112 of the vascular access 108. Ideally, blood flow is balanced between the vascular access 108 and the arteries feeding the extremities, in FIG. 1, the ulnar artery 114 and the radial artery 116.

In general, a minimum blood flow rate of approximately 500 ml/min is required from the vascular access, but no more than 1000 ml/min is needed in order to perform dialysis. However, most vascular accesses provide a blood flow rate of 600-3000 ml/min. Blood flow rates that are too low are common and treatable with endovascular techniques including angioplasty, thrombectomy, and stenting to restore higher blood flow rates. However, blood flow rates that are too high (high flow fistulas and grafts), while capable of providing good flow for dialysis, can result in complications.

One such complication is the vascular “steal phenomenon”, which occurs when excessive blood flows into the vascular access, and is therefore diverted away from the patient's extremities, most commonly the hand. This complication is illustrated in FIG. 2, where the number of arrows reflect blood flow rates. Referring to FIG. 2, excess blood flow rates are seen in the vascular access 108, while inadequate blood flow rates are seen in the ulnar artery 114 and the radial artery 116. Inadequate blood flow rates can cause pain, loss of motor function, loss of digits, and at times loss of the entire hand due to ischemia.

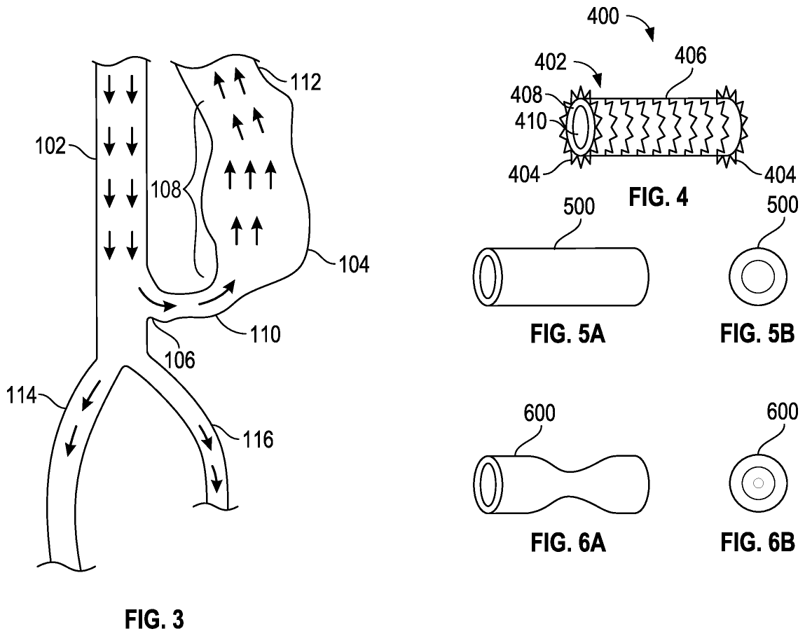

Another complication is the formation of aneurysms due to venous hypertension, as illustrated in FIG. 3. This complication involves dilatation of the vascular access 108 due to excessive inflow (flow coming into the access 108 from the artery 102) relative to the size of the venous end of the access 112 (flow leaving the access 108 and ultimately returning to the right side of the heart). Particularly with fistulas located in the upper arm, a “megafistula” can develop which is a large serpentine thick-walled vascular structure. These are cosmetically problematic but also not too infrequently can rupture resulting in exsanguination and death.

Another complication is high output cardiac failure. In this complication, vascular accesses with high flow (e.g., 2000-3000 ml/min), particularly in patients with underlying heart disease, can result in excess stress on the heart, causing progressive heart failure and death.

As described above, current techniques to increase blood flow in the dialysis vascular access are endovascular and percutaneous, do not require open surgery, and can be safely performed in the outpatient setting. However, the only existing treatments to reduce blood flow in the dialysis vascular access currently require surgery. Like most surgical procedures, these procedures require incisions and anesthesia, and incur risks that come with surgery, for example including excess bleeding, infection, and complications from anesthesia. The disclosed technologies present a novel, first of its kind, endovascular percutaneous technique to solve the problem of high flow dialysis vascular access without the need for surgery, and which can be safely performed in an outpatient setting.

Embodiments of the disclosed technologies provide a flow diverting device (FDD) and method for dialysis vascular access. The FDD may be placed percutaneously using existing endovascular techniques and angiography to deploy the FDD on the arterial end of the vascular access. The FDD may use existing stent technology as the outer framework or matrix. The FDD may include a covered stent with an inner luminal structure to divert flow away from the vascular access and towards the native artery of the patient. Using existing stent technology, specifically the struts of the stent at both ends, the FDD anchors to the lumen of the vascular access. The inner lumen of the FDD acts as a physical barrier to discourage high flow into the vascular access and encourage more blood flow to the distal artery, and thus toward the patient's extremity.

FIG. 4 is a perspective view of an FDD 400 according to some embodiments of the disclosed technologies. The FDD 400 may include a tubular vascular stent 402. The tubular vascular stent 402 may be fabricated in a conventional manner from conventional materials. The tubular vascular stent 402 may include struts 404 at one or both ends. The struts 404 may serve to anchor the FDD 400 within the lumen of the vascular access. The struts 404 may be fabricated in a conventional manner from conventional materials. The tubular vascular stent 402 may include one or more radio-opaque markers at one or both ends (not shown).

The FDD 400 may include a tubular stent cover 406, wherein the vascular stent 402 is disposed within the tubular stent cover 406. The tubular stent cover 406 may be made of conventional materials. For example, the tubular stent cover 406 may be a coating made of PTFE.

The FDD 400 may include a flow restrictor 408 disposed within the tubular vascular stent 402. The flow restrictor 408 may include an inner longitudinal lumen 410 that restricts the blood flow rate through the vascular access to a desired range or percentage.

The disclosed flow restrictors may be fabricated of conventional materials. In some examples, the flow restrictors may be made of polyurethane or another polymer. In some examples, the flow restrictors may be made of animal tissue such as bovine or porcine tissue similar to that used in bioprosthetic heart valves.

The inner lumen of the disclosed flow restrictor may have different shapes. In some examples, the diameter of the inner lumen may be uniform. FIG. 5A is a perspective view of a flow restrictor 500 having an inner lumen of uniform diameter. FIG. 5B is a cross-sectional view of the flow restrictor 500 of FIG. 5A.

In some examples, the diameter of the inner lumen may be non-uniform. FIG. 6A is a perspective view of a flow restrictor 600 having an inner lumen of non-uniform diameter. FIG. 6B is a cross-sectional view of the flow restrictor 600 of FIG. 6A. In the flow restrictor 600, the diameter of the inner lumen at the ends of the inner lumen is greater than the diameter of the inner lumen between the ends of the inner lumen. However, other arrangements are contemplated.

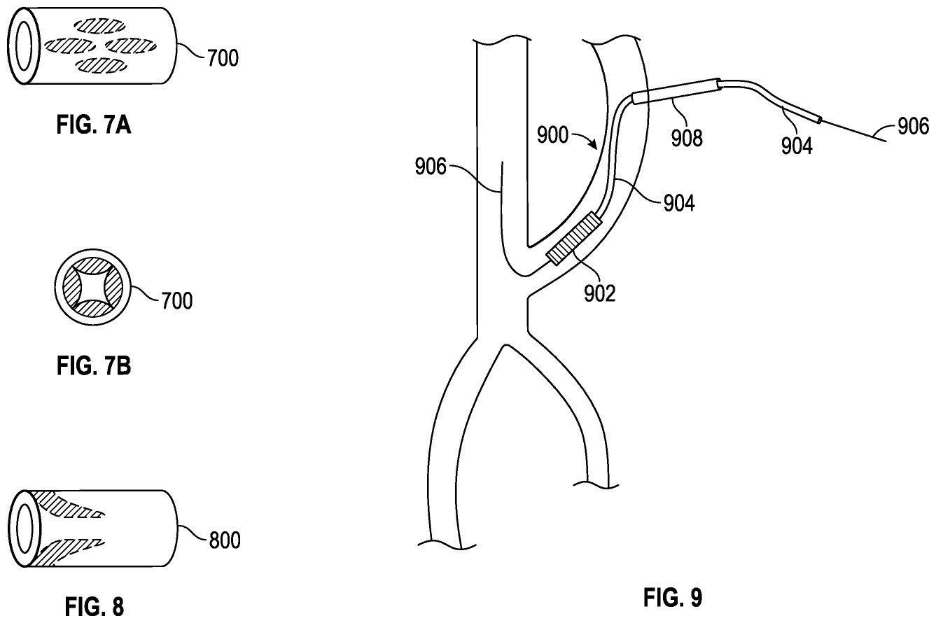

In some examples, one or more projections may extend into the inner lumen. FIG. 7A is a perspective view of a flow restrictor 700 having a scalloped luminal structure. FIG. 7B is a cross-sectional view of the flow restrictor 700 of FIG. 7A. FIG. 8 is a perspective view of a flow restrictor 800 having a venous valve luminal structure. However, other numbers and shapes of luminal projections are contemplated.

Different lengths and diameters of the FDD may be employed. The outer diameter of the FDD device size may be selected to fit the FDD to the appropriate vessel diameter, similar to how a venous stent is sized, erring on the side of oversizing to avoid the device from dislodging from the wall of the vessel. For example, the outer diameter may range from 6 mm to 24 mm. Various different inner luminal lengths and diameters may be selected depending on the degree of flow diversion desired. This degree may be represented as a percentage of unobstructed blood flow (e.g., a 60% degree of flow diversion would restrict blood flow by 60%).

Devices for deploying the FDD may be similar to conventional stent deployment devices. For example, the FDD may be compressed and pre-loaded on the shaft of the deployment device. FIG. 9 illustrates a portion of a deployment device 900 according to some embodiments of the disclosed technologies. Referring to FIG. 9, the deployment device 900 may include a FDD 902. The deployment device 900 may include a deployment catheter 904 connected to the FDD 902. The deployment device 900 may include a guidewire 906 disposed within the deployment catheter 904. For example, the guidewire 906 may be a 0.035″ guidewire. The deployment device 900 may include an angiographic sheath 908. The deployment catheter 904 may be disposed within the angiographic sheath 908.

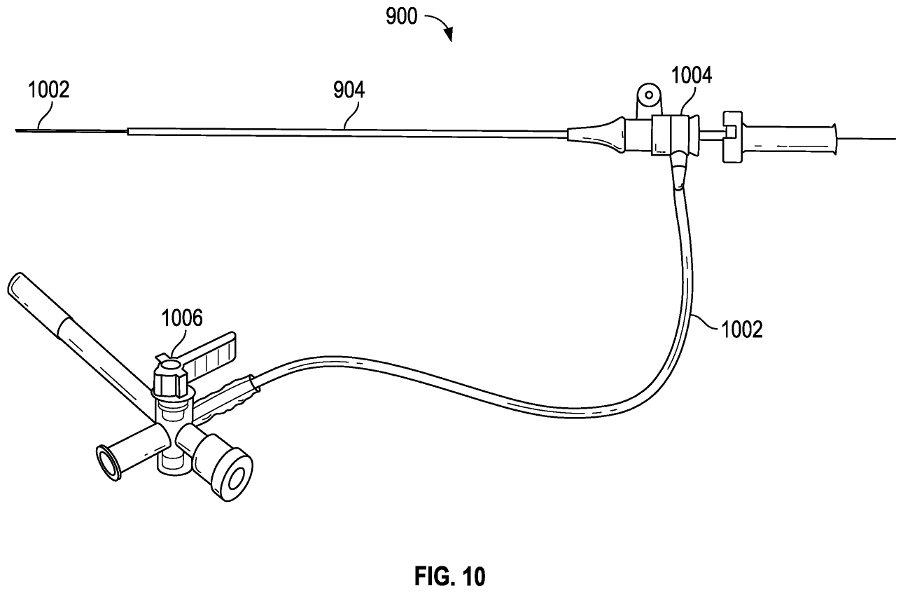

FIG. 10 illustrates additional components of the deployment device 900 of FIG. 9 according to some embodiments of the disclosed technologies. Referring to FIG. 10, the deployment device 900 may include a side tube 1002, a connector 1004 coupling the deployment catheter 904 and a first end of the side tube 1002, and a three-way stopcock 1006 coupled to a second end of the side tube 1002.

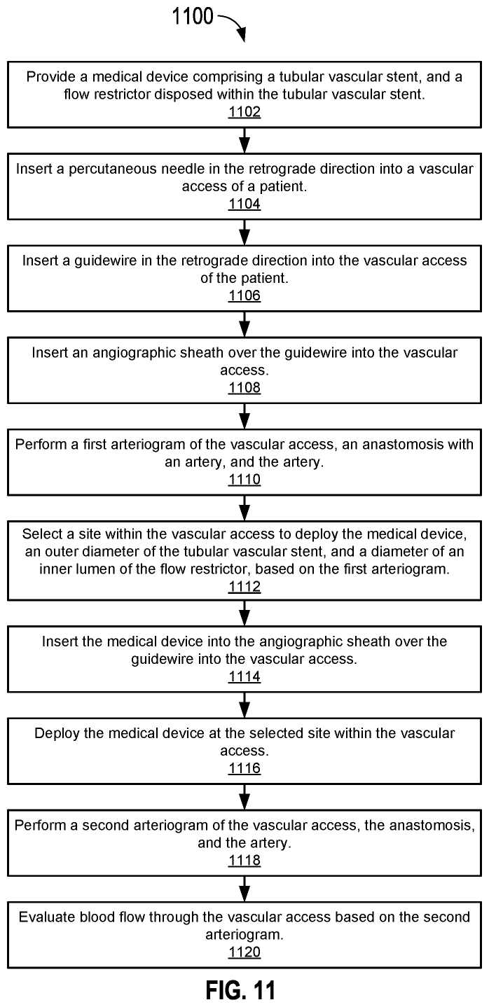

FIG. 11 is a flowchart illustrating a process 1100 for deploying the FDD according to some embodiments of the disclosed technologies. The elements of the process 1100 are presented in one arrangement. However, it should be understood that one or more elements of the process may be performed in a different order, in parallel, omitted entirely, and the like. Furthermore, the process 1100 may include other elements in addition to those presented.

Referring to FIG. 11, the process 1100 may include providing a medical device comprising a tubular vascular stent, and a flow restrictor disposed within the tubular vascular stent, at 1102. For example, the tubular vascular stent and flow restrictor may be the FDD 400 of FIG. 4.

Referring to FIG. 11, the process 1100 may include inserting a percutaneous needle in the retrograde direction into a vascular access of a patient, at 1104. For example, the vascular access may include a vein of the patient, as illustrated in FIG. 9. Referring again to FIG. 11, the process 1100 may include inserting a guidewire in the retrograde direction into the vascular access of a patient, at 1106.

Referring again to FIG. 11, the process 1100 may include inserting an angiographic sheath over the guidewire into the vascular access, at 1108, for example as illustrated in FIG. 9.

Referring again to FIG. 11, the process 1100 may include performing a first arteriogram of the vascular access, an anastomosis with an artery, and the artery, at 1110, for example as illustrated in FIG. 9. Performing the arteriogram may include inserting the guidewire 906 in the retrograde direction into the vascular access and into an artery corresponding to the vein through an anastomosis between the artery and the vein such that a tip of the guidewire 906 is located in the artery and proximal to the anastomosis. A catheter 904 may be inserted along the guidewire 906 such that a tip of the catheter is located in the artery and proximal to the anastomosis. Deploying arteriographic fluid in the artery and proximal to the anastomosis allows the fluid to flow through the artery, the anastomosis, the vascular access, and the distal arteries, providing a complete representation of the relevant blood flows.

Referring again to FIG. 11, the process 1100 may include selecting a site within the vascular access to deploy the medical device, an outer diameter of the tubular vascular stent, and a diameter of an inner lumen of the flow restrictor, based on the first arteriogram, at 1112. The process 1100 may include inserting the medical device into the angiographic sheath over the guidewire into the vascular access, at 1114. The process 1100 may include deploying the FDD at the selected site within the vascular access, at 1116. Conventional stent deployment equipment and techniques may be employed.

After deploying the FDD, the process 1100 may include performing a second arteriogram of the vascular access, the anastomosis, and the artery, at 1118. The second arteriogram may be performed in the same manner as the first arteriogram. The process 1100 may include evaluating blood flow through the vascular access based on the second arteriogram, at 1120. Based in the evaluation, blood flow may be increased or decreased further. For example, to increase blood flow, an angioplasty balloon may be inflated within the FDD to increase the diameter of its inner lumen. To decrease blood flow further, a second FDD may be placed in series the first FDD.

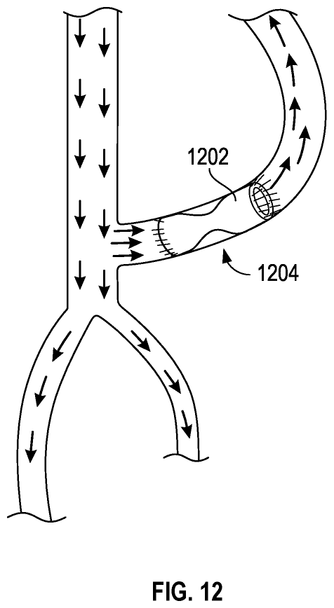

Finally, the guidewire and sheath may then be removed. FIG. 12 illustrates a FDD 1202 deployed within the vascular access 1204 according to some embodiments of the disclosed technologies.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

What is claimed is:

1. A medical device comprising:
a tubular vascular stent; and
a flow restrictor disposed within the tubular vascular stent.

2. The vascular medical device of claim 1, further comprising:
a tubular stent cover, wherein the vascular stent is disposed within the tubular stent cover.

3. The vascular medical device of claim 1, wherein the tubular stent comprises:
one or more anchor struts formed on at least one end of the tubular stent.

4. The vascular medical device of claim 1, wherein the flow restrictor comprises:
a longitudinal lumen, wherein a diameter of the lumen is uniform.

5. The vascular medical device of claim 1, wherein the flow restrictor comprises:
a longitudinal lumen, wherein a diameter of the lumen is non-uniform.

6. The vascular medical device of claim 1, wherein the flow restrictor comprises:
a longitudinal lumen, wherein a diameter of the lumen at an end of the lumen is greater than a diameter of the lumen between the ends of the lumen.

7. The vascular medical device of claim 1, wherein the flow restrictor comprises:
a longitudinal lumen; and
one or more projections extending into the lumen.

8. The vascular medical device of claim 1, further comprising:
one or more radio-opaque markers.

9. A method comprising:
providing a medical device, the medical device comprising:
a tubular vascular stent, and
a flow restrictor disposed within the tubular vascular stent; and
deploying the medical device at a site within a vascular access of a patient.

10. The method of claim 9, wherein the vascular access is to a vein of the patient, and wherein disposing the medical device within a vascular access of a patient comprises:
inserting a percutaneous needle in the retrograde direction into the vascular access;
inserting a guidewire in the retrograde direction into the vascular access;
inserting an angiographic sheath over the guidewire into the vascular access;
inserting the medical device into the angiographic sheath over the guidewire into the vascular access;
removing the sheath; and
removing the guidewire.

11. The method of claim 10, further comprising:
performing a first arteriogram of the vascular access prior to deploying the medical device;
selecting the site within the vascular access to deploy the medical device, an outer diameter of the tubular vascular stent, and a diameter of an inner lumen of the flow restrictor, based on the first arteriogram.

12. The method of claim 11, further comprising:
performing a second arteriogram subsequent to deploying the medical device; and
evaluating blood flow through the vascular access, and blood flow through the artery distal to the anastomosis, based on the second arteriogram.

13. The method of claim 11, wherein performing a first arteriogram comprises:
inserting the guidewire in the retrograde direction into the vascular access and into an artery corresponding to the vein through an anastomosis between the artery and the vein such that a tip of the guidewire is located in the artery and proximal to the anastomosis;
inserting a catheter along the guidewire such that a tip of the catheter is located in the artery and proximal to the anastomosis; and
deploying arteriographic fluid in the artery and proximal to the anastomosis.

14. A medical device comprising:
a tubular vascular stent;
one or more anchor struts formed on at least one end of the tubular stent;
a flow restrictor disposed within the tubular vascular stent; and
a tubular stent cover, wherein the vascular stent is disposed within the tubular stent cover.

15. The vascular medical device of claim 14, wherein the flow restrictor comprises:
a longitudinal lumen, wherein a diameter of the lumen is uniform.

16. The vascular medical device of claim 14, wherein the flow restrictor comprises:
a longitudinal lumen, wherein a diameter of the lumen is non-uniform.

17. The vascular medical device of claim 14, wherein the flow restrictor comprises:
a longitudinal lumen, wherein a diameter of the lumen at an end of the lumen is greater than a diameter of the lumen between the ends of the lumen.

18. The vascular medical device of claim 14, wherein the flow restrictor comprises:
a longitudinal lumen; and
one or more projections extending into the lumen.

19. The vascular medical device of claim 14, further comprising:
one or more radio-opaque markers.
